SEQUENCE LISTING (12 sequences)

SEQ ID NO: 1 - DNA, 1414 bp, Homo sapiens
gctcccggggacattctaaccgccgccaggtcccgccgcctctcgccccgctattaataccggcggcccgggaggggggcgcagcacgcgccgcgcagccatggggaggctgctggccttagtggtcggcgcggcactggtgtcctcagcctgcgggggctgcgtggaggtggactcggagaccgaggccgtgtatgggatgaccttcaaaattctttgcatctcctgcaagcgccgcagcgagaccaacgctgagaccttcaccgagtggaccttccgccagaagggcactgaggagtttgtcaagatcctgcgctatgagaatgaggtgttgcagctggaggaggatgagtgcttcgagggccgcgtggtgtggaatggcagccggggcaccaaagacctgcaggatctgtctatcttcatcaccaatgtcacctacaaccactcgggcgactacgagtgccacgtctaccgcctgctcttcttcgaaaactacgagcacaacaccagcgtcgtcaagaagatccacattgaggtagtggacaaagccaacagagacatggcatccatcgtgtctgagatcatgatgtatgtgctcattgtggtgttgaccatatggctcgtggcagagatgatttactgctacaagaagatcgctgccgccacggagactgctgcacaggagaatgcctcggaatacctggccatcacctctgaaagcaaagagaactgcacgggcgtccaggtggccgaatagccctggccctgggccccgcctcaaggaagagccagccgtaatggggactctccaggcaccgcctgcccccagcgtgggggtggccactcctgggccccagaaagcctcagagtcctgccgacggagccactggggtgggagggggcagggggcttggctcgcacccccactttcgcctcctccagctcctgccccgccggccgcgcaccgccatgcatgatgggtaaagcaatactgccgctgcccccaccctgcttctgctgcctgtttggggaggggggcggtgaggtgggggcagcggccccgcacccctcctccttgctgatttgcacacattggccgcttcagacacgcacttctggggccagcccctccccgcctcctccctgcctggcggcaggggtcgcgatgatgggctggagcagtttggggcagggggttctgggacccactccgactccccctccccggcatcatttcccctcccgcttcctccggctggacctggggtcccccctccctgtaatgcactcctgccccggcccaacctcgccctctctcaccagccttgaactgtggccacctagaaaggggcccattcagcctcgtctctttacagaagtagttttgttcatgaaataaagactcttggacttg

SEQ ID NO: 2 - protein, 218 aa, Homo sapiens
MGRLLALVVGAALVSSACGGCVEVDSETEAVYGMTFKILCISCKRRSETNAETFTEWTFRQKGTEEFVKILRYENEVLQLEEDECFEGRVVWNGSRGTKDLQDLSIFITNVTYNHSGDYECHVYRLLFFENYEHNTSVVKKIHIEVVDKANRDMASIVSEIMMYVLIVVLTIWLVAEMIYCYKKIAAATETAAQENASEYLAITSESKENCTGVQVAE

SEQ ID NO: 3 - DNA, 20 bp, Homo sapiens
cgcctctcgccccgctatta

SEQ ID NO: 4 - DNA, 19 bp, Homo sapiens
ctcccgccgccccgccgtg

SEQ ID NO: 5 - DNA, 20 bp, Homo sapiens
cctgacctgagcctgctgtc

SEQ ID NO: 6 - DNA, 19 bp, Homo sapiens
tgccctcccatgccgttaa

SEQ ID NO: 7 - DNA, 19 bp, Homo sapiens
ccttcccctccctggctac

SEQ ID NO: 8 - DNA, 20 bp, Homo sapiens
ggcaggcagcacccgactca

SEQ ID NO: 9 - DNA, 20 bp, Homo sapiens
cagcctgggctaccccctta

SEQ ID NO: 10 - DNA, 20 bp, Homo sapiens
ccctgggtgccctcccacct

SEQ ID NO: 11 - DNA, 20 bp, Homo sapiens
cggtctgatgatggggtcac

SEQ ID NO: 12 - DNA, 20 bp, Homo sapiens
ttacggctggctcttccttg

(19) United States
(12) Patent Application Publication
(10) Pub. No.: US 2004/0191791 A1
(43) Pub. Date: Sep. 30, 2004

(54) Novel mutation

(76) Inventors: Robyn Heather Wallace, Memphis, TN (US); John Charles Mulley, Firle (AU); Ingrid Eileen Scheffer, Malvern East (AU)

Correspondence Address: Arles A Taylor Jr, Jenkins Wilson & Taylor, Suite 1400 University Tower, 3100 Tower Boulevard, Durham, NC, 27707, US

(21) Appl. No.: 10/477,272

(22) PCT Filed: May 9, 2002
(86) PCT No.: PCT/AU02/00581

(30) Foreign Application Priority Data
May 10, 2001 (AU) PR 4922

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

An isolated nucleic acid molecule encoding a mutant mammalian beta-1 subunit of a voltage-gated sodium channel wherein a mutation event has occurred and said mutation event disrupts the functioning of an assembled sodium channel so as to produce an epilepsy phenotype, with the proviso that said mutation event is not one which results in a C121W substitution in the encoded polypeptide.

### TECHNICAL FIELD

The present invention is concerned with a new mutation in the SCN1B gene that is involved in epilepsy and, more particularly, with a new SCN1B gene mutation associated with generalised epilepsy with febrile seizures plus (GEFS+).

### BACKGROUND ART

Epilepsies constitute a diverse collection of brain disorders that affect about 3% of the population at some time in their lives (Annegers, 1996). An epileptic seizure can be defined as an episodic change in behaviour caused by the disordered firing of populations of neurons in the central nervous system. This results in varying degrees of involuntary muscle contraction and often a loss of consciousness. Epilepsy syndromes have been classified into more than 40 distinct types based upon characteristic symptoms, types of seizure, cause, age of onset and EEG patterns (Commission on Classification and Terminology of the International League Against Epilepsy, 1989). However the single feature that is common to all syndromes is the persistent increase in neuronal excitability that is both occasionally and unpredictably expressed as a seizure.

A genetic contribution to the aetiology of epilepsy has been estimated to be present in approximately 40% of affected individuals (Gardiner, 2000). As epileptic seizures may be the end-point of a number of molecular aberrations that ultimately disturb neuronal synchrony, the genetic basis for epilepsy is likely to be heterogeneous. There are over 200 Mendelian diseases which include epilepsy as part of the phenotype. In these diseases, seizures are symptomatic of underlying neurological involvement such as disturbances in brain structure or function. In contrast, there are also a number of “pure” epilepsy syndromes in which epilepsy is the sole manifestation in the affected individuals. These are termed idiopathic and account for over 60% of all epilepsy cases.

Idiopathic epilepsies have been further divided into partial and generalized sub-types. Partial (focal or local) epileptic fits arise from localized cortical discharges, so that only certain groups of muscles are involved and consciousness may be retained (Sutton, 1990). However, in generalized epilepsy, EEG discharge shows no focus such that all subcortical regions of the brain are involved. Although the observation that generalized epilepsies are frequently inherited is understandable, the mechanism by which genetic defects, presumably expressed constitutively in the brain, give rise to partial seizures is less clear.

The idiopathic generalized epilepsies (IGE) are the most common group of inherited human epilepsies. Two broad groups of IGE are now known—the classical idiopathic generalized epilepsies (Commission on Classification and Terminology of the International League Against Epilepsy, 1989) and the newly recognized genetic syndrome of generalized epilepsy with febrile seizures plus (GEFS+) (Scheffer and Berkovic, 1997; Singh et al. 1999). The classical IGEs are divided into a number of clinically recognizable but overlapping sub-syndromes including childhood absence epilepsy (CAE), juvenile absence epilepsy, juvenile myoclonic epilepsy etc (Commission on Classification and Terminology of the International League Against Epilepsy, 1989; Roger et al. 1992). The sub-syndromes are identified by age of onset and the pattern of seizure types (absence, myoclonus and tonic-clonic). Some patients, particularly those with tonic-clonic seizures alone do not fit a specifically recognized sub-syndrome. Arguments for regarding these as separate syndromes, yet recognizing that they are part of a neurobiological continuum, have been presented previously (Berkovic et al. 1987; 1994; Reutens and Berkovic, 1995).

Generalised epilepsy with febrile seizures plus (GEFS+; MIN 604236) was first described in 1997 (Scheffer & Berkovic, 1997) and is now recognised as a common epilepsy syndrome (Singh et al. 1999; Baulac et al. 1999; Peiffer et al. 1999; Scheffer et al. 2000). Although GEFS+is familial, it was initially difficult to recognise it as a distinct syndrome due to clinical heterogeneity within each family. The common phenotypes are typical febrile seizures and febrile seizures plus (FS+); FS+differs from typical febrile seizures in that the attacks with fever continue beyond 6 years and/or include afebrile tonic-clonic seizures. Less common phenotypes include FS+associated with absences, myoclonic seizures or atonic seizures and even more severe syndromes such as myoclonicastatic epilepsy. That such phenotypic diversity could be associated with the segregation of a mutation in a single gene was established with the identification of a mutation in the voltage-gated sodium channel beta-1 subunit gene (SCN1B) (Wallace et al. 1998). The mutation (C121W) changes a conserved cysteine residue, disrupting a putative disulfide bridge with in vitro loss of function of the SCN1B subunit. Without a functional SCN1B subunit the rate of inactivation of sodium channel alpha subunits decreases, which may cause increased sodium influx, resulting in a more depolarised membrane potential and hyperexcitability.

In additional studies, four GEFS+families were mapped to chromosome 2q (Baulac et al. 1999; Moulard et al. 1999; Peiffer et al. 1999; Lopes-Cendes et al. 2000). Recently, mutations in the neuronal voltage gated sodium channel alpha-1 (SCN1A) subunit which maps to chromosome 2q were described in GEFS+families (Wallace et al. 2001). The mutations (D188V, V1353L and 11656M) are all located in highly conserved residues situated adjacent or within the membrane spanning segments of the subunit. Mutations in SCN1A have also been identified in additional families (Escayg et al. 2000). These mutations (T875M and R1648H) are located in highly conserved S4 transmembrane segments of the channel which are known to have a role in channel gating. Functional studies of the R1648H mutation in the same conserved region of SCN4A (R1460H) have shown that the time-course of inactivation of the mutant channels is slightly slowed and the recovery from inactivation is accelerated when compared to wild-type channels (Alekov et al. 2000). A combination of the subtle changes in activation and fast inactivation are therefore sufficient to cause epilepsy. GEFS+is clearly a common complex disorder, with a strong genetic basis, incomplete penetrance and genetic and phenotypic heterogeneity. Febrile seizures occur in 3% of the population, and thus this phenotype may occur sporadically in GEFS+families, in addition to occurring as a result of the GEFS+gene (Wallace et al 1998). Also, although some families segregate an autosomal dominant gene of major effect, in many cases clinical genetic evidence, such as bilineality, suggests that for some small families the disorder is multifactorial (Singh et al 1999).

Despite the rarity of SCN1A and SCN1B mutations in GEFS+, a novel mutation in the SCN1B gene has been identified in affected families. The frequency of involvement of the SCN1 genes in epilepsy may therefore be greater than first thought.

### DISCLOSURE OF THE INVENTION

The present inventors have identified a novel mutation in the beta-1 subunit (SCN1B) of the voltage-gated sodium channel that is associated with epilepsy, in particular generalized epilepsy with febrile seizures plus (GEFS+).

In one embodiment, the present invention provides an isolated mammalian nucleic acid molecule encoding a mutant beta-1 subunit of a voltage-gated sodium channel wherein a mutation event has occurred and said mutation event disrupts the functioning of an assembled sodium channel so as to produce an epilepsy phenotype, with the proviso that said mutation event is not one which results in a C121W substitution in the encoded polypeptide. In particular, the mutation lies in the extracellular loop of the amino terminal domain of SCN1B at amino acid position 85.

In one form of the invention, the mutation is in exon 3 of SCN1B and results in the replacement of an arginine residue with a cysteine residue at amino acid position 85. The R85C mutation occurs as a result of a C to T nucleotide substitution at position 253 of the SCN1B coding sequence as illustrated in SEQ ID NO: 1. Preferably the mutation creates a phenotype of generalized epilepsy with febrile seizures plus.

The nucleotide sequences of the present invention can be engineered using methods accepted in the art for a variety of purposes. These include, but are not limited to, modification of the cloning, processing, and/or expression of the gene product. PCR reassembly of gene fragments and the use of synthetic oligonucleotides allow the engineering of the nucleotide sequences of the present invention. For example, oligonucleotide-mediated site-directed mutagenesis can introduce further mutations that create new restriction sites, alter expression patterns and produce splice variants etc.

As a result of the degeneracy of the genetic code, a number of polynucleotide sequences, some that may have minimal similarity to the polynucleotide sequences of any known and naturally occurring gene, may be produced. Thus, the invention includes each and every possible variation of a polynucleotide sequence that could be made by selecting combinations based on possible codon choices. These combinations are made in accordance with the standard triplet genetic code as applied to the polynucleotide sequences of the present invention, and all such variations are to be considered as being specifically disclosed.

The DNA molecules of this invention include cDNA, genomic DNA, synthetic forms, and mixed polymers, both sense and antisense strands, and may be chemically or biochemically modified, or may contain non-natural or derivatised nucleotide bases as will be appreciated by those skilled in the art. Such modifications include labels, methylation, intercalators, alkylators and modified linkages. In some instances it may be advantageous to produce nucleotide sequences possessing a substantially different codon usage than that of the polynucleotide sequences of the present invention. For example, codons may be selected to increase the rate of expression of the peptide in a particular prokaryotic or eukaryotic host corresponding with the frequency that particular codons are utilized by the host. Other reasons to alter the nucleotide sequence without altering the encoded amino acid sequences include the production of RNA transcripts having more desirable properties, such as a greater half-life, than transcripts produced from the naturally occurring mutated sequence.

The invention also encompasses production of DNA sequences of the present invention entirely by synthetic chemistry. Synthetic sequences may be inserted into expression vectors and cell systems that contain the necessary elements for transcriptional and translational control of the inserted coding sequence in a suitable host. These elements may include regulatory sequences, promoters, 5′ and 3′ untranslated regions and specific initiation signals (such as an ATG initiation codon and Kozak consensus sequence) which allow more efficient translation of sequences encoding the polypeptides of the present invention. In cases where the complete coding sequence, including the initiation codon and upstream regulatory sequences, are inserted into the appropriate expression vector, additional control signals may not be needed. However, in cases where only coding sequence, or a fragment thereof, is inserted, exogenous translational control signals as described above should be provided by the vector. Such signals may be of various origins, both natural and synthetic. The efficiency of expression may be enhanced by the inclusion of enhancers appropriate for the particular host cell system used (Scharf et al., 1994).

The invention also includes nucleic acid molecules that are the complements of the sequences described herein.

The present invention allows for the preparation of purified polypeptides or proteins from the polynucleotides of the present invention, or variants thereof. In order to do this, host cells may be transformed with a DNA molecule as described above. Typically said host cells are transfected with an expression vector comprising a DNA molecule according to the invention. A variety of expression vector/host systems may be utilized to contain and express sequences encoding polypeptides of the invention. These include, but are not limited to, microorganisms such as bacteria transformed with plasmid or cosmid DNA expression vectors; yeast transformed with yeast expression vectors; insect cell systems infected with viral expression vectors (e.g., baculovirus); or mouse or other animal or human tissue cell systems. Mammalian cells can be used to express a protein using various expression vectors including plasmid, cosmid and viral systems such as a vaccinia virus expression system. The invention is not limited by the host cell employed.

The polynucleotide sequences, or variants thereof, of the present invention can be stably expressed in cell lines to allow long term production of recombinant proteins in mammalian systems. Sequences encoding the polypeptides of the present invention can be transformed into cell lines using expression vectors which may contain viral origins of replication and/or endogenous expression elements and a selectable marker gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

The protein produced by a transformed cell may be secreted or retained intracellularly depending on the sequence and/or the vector used. As will be understood by those of skill in the art, expression vectors containing polynucleotides which encode a protein may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of a “prepro” form of the protein may also be used to specify protein targeting, folding, and/or activity. Different host cells having specific cellular machinery and characteristic mechanisms for post-translational activities (e.g., CHO or HeLa cells), are available from the American Type Culture Collection (ATCC) and may be chosen to ensure the correct modification and processing of the foreign protein.

When large quantities of the gene are needed, such as for antibody production, vectors which direct high levels of expression of this protein may be used, such as those containing the T5 or T7 inducible bacteriophage promoter. The present invention also includes the use of the expression systems described above in generating and isolating fusion proteins which contain important functional domains of the protein. These fusion proteins are used for binding, structural and functional studies as well as for the generation of appropriate antibodies.

In order to express and purify the protein as a fusion protein, the appropriate polynucleotide sequences of the present invention are inserted into a vector which contains a nucleotide sequence encoding another peptide (for example, glutathionine-s-transferase). The fusion protein is expressed and recovered from prokaryotic or eukaryotic cells. The fusion protein can then be purified by affinity chromatography based upon the fusion vector sequence. The desired protein is then obtained by enzymatic cleavage of the fusion protein.

Fragments of polypeptides of the present invention may also be produced by direct peptide synthesis using solid-phase techniques. Automated synthesis may be achieved by using the ABI 431A Peptide Synthesizer (Perkin-Elmer). Various fragments of this protein may be synthesized separately and then combined to produce the full-length molecule.

According to still another aspect, the invention provides an isolated mammalian polypeptide, said polypeptide being a mutant beta-1 subunit of a voltage-gated sodium channel, wherein a mutation event has occurred and said mutation event disrupts the functioning of an assembled sodium channel so as to produce an epilepsy phenotype, with the proviso that said mutation event is not a C121W substitution. In particular, the mutation lies in the extracellular loop of the amino terminal domain of SCN1B at amino acid position 85.

In one form of the invention, the mutation event is a substitution in which an arginine residue is replaced with a cysteine residue at amino acid position 85 of the SCN1B protein as illustrated in SEQ ID NO: 2.

According to still another aspect of the invention, there is provided an isolated polypeptide complex, said polypeptide complex being an assembled mammalian voltage-gated sodium channel, wherein a mutation event has taken place in the beta-1 subunit and said mutation event disrupts the functioning of the assembled sodium channel. Preferably, there is a mutation at amino acid position 85 of the SCN1B subunit of the channel.

In a further aspect, the mutation is an R85C mutation in SCN1B.

According to another aspect of the present invention there is provided a method of preparing a polypeptide, said polypeptide being a mutant SCN1B subunit of a mammalian voltage-gated sodium channel, comprising the steps of:

(1) culturing host cells transfected with an expression vector comprising a nucleic acid molecule as described above under conditions effective for polypeptide production; and

(2) harvesting the mutant SCN1B subunit.

The mutant SCN1B subunit may also be allowed to assemble with other subunits of the sodium channel that may be co-expressed by the cell, whereby the assembled mutant sodium channel is harvested.

According to still another aspect of the invention there is provided a polypeptide which is the product of the process described above.

Substantially purified protein or fragments thereof can then be used in further biochemical analyses to establish secondary and tertiary structure for example by X-ray crystallography of crystals of the proteins or by nuclear magnetic resonance (NMR). Determination of structure allows for the rational design of pharmaceuticals to interact with the mutated sodium channel, alter the overall sodium channel protein charge configuration or charge interaction with other proteins, or to alter its function in the cell.

It will be appreciated that, having identified a new mutation involved in epilepsy in SCN1B, the mutant sodium channel beta-1 subunit of the present invention will enable therapeutic methods for the treatment of epilepsy including, but not restricted to, generalised epilepsy with febrile seizures plus as well as other disorders associated with sodium channel dysfunction. The mutant SCN1B subunit of the present invention will also be useful in diagnostic applications to screen for and detect the presence of the mutated gene or gene product in individuals affected by disorders as described above.

Therapeutic Applications

According to one aspect of the invention there is provided a method of treating epilepsy, as well as other disorders associated with sodium channel dysfunction, comprising administering a selective antagonist, agonist or modulator of the sodium channel when it contains a mutation as described above, more particularly, a mutation at amino acid position 85 of an SCN1B subunit comprising the channel.

In still another aspect of the invention there is provided the use of a selective antagonist, agonist or modulator of the sodium channel when it contains a mutation as described above, more particularly, a mutation at amino acid position 85 of an SCN1B subunit comprising the channel, said mutation being causative of a disorder including epilepsy in the manufacture of a medicament for the treatment of the disorder.

In one aspect of the invention a suitable agonist, antagonist or modulator will restore wild-type function to sodium channels containing SCN1B mutations that form part of this invention or will negate the effects a mutant channel has on cell function.

Using methods well known in the art, a mutant sodium channel may be used to produce antibodies specific for the mutant channel that is causative of the disease or to screen libraries of pharmaceutical agents to identify those that bind the mutant sodium channel.

In one aspect, an antibody, which specifically binds to a mutant sodium channel or mutant SCN1B subunit of the invention, may be used directly as an antagonist or modulator, or indirectly as a targeting or delivery mechanism for bringing a pharmaceutical agent to cells or tissues that express the mutant sodium channel.

In a still further aspect of the invention there is provided an antibody which is immunologically reactive with a polypeptide as described above, but not with a wild-type sodium channel or SCN1B subunit thereof.

In particular, there is provided an antibody to an assembled sodium channel which contains an SCN1B subunit mutation of the invention that is causative of a disorder. Such antibodies may include, but are not limited to, polyclonal, monoclonal, chimeric, and single chain antibodies as would be understood by the person skilled in the art.

For the production of antibodies, various hosts including rabbits, rats, goats, mice, humans, and others may be immunized by injection with a polypeptide as described or with any fragment or oligopeptide thereof which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum.

It is preferred that the oligopeptides, peptides, or fragments used to induce antibodies to the mutant sodium channel have an amino acid sequence consisting of at least 5 amino acids, and, more preferably, of at least 10 amino acids. It is also preferable that these oligopeptides, peptides, or fragments are identical to a portion of the amino acid sequence of the natural protein and contain the entire amino acid sequence of a small, naturally occurring molecule. Short stretches of sodium channel amino acids may be fused with those of another protein, such as KLH, and antibodies to the chimeric molecule may be produced.

Monoclonal antibodies to a mutant sodium channel may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler et al., 1975; Kozbor et al., 1985; Cote et al., 1983; Cole et al., 1984).

Antibodies may also be produced by inducing in vivo production in the lymphocyte population or by screening immunoglobulin libraries or panels of highly specific binding reagents as disclosed in the literature. (For example, see Orlandi et al., 1989; Winter et al., 1991).

Antibody fragments which contain specific binding sites for a mutant sodium channel may also be generated. For example, such fragments include, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (For example, see Huse et al., 1989).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between a sodium channel and its specific antibody. A two-site, monoclonal-based immunoassay utilizing antibodies reactive to two non-interfering sodium channel epitopes is preferred, but a competitive binding assay may also be employed.

In a further aspect of the invention there is provided a method of treating epilepsy, as well as other disorders associated with sodium channel dysfunction, comprising administering an isolated DNA molecule which is the complement (antisense) of any one of the DNA molecules described above and which encodes an RNA molecule that hybridizes with the mRNA encoding a mutant sodium channel beta-1 subunit of the invention, to a subject in need of such treatment.

Typically, a vector expressing the complement of the polynucleotides of the invention may be administered to a subject in need of such treatment. Antisense strategies may use a variety of approaches including the use of antisense oligonucleotides, injection of antisense RNA, ribozymes, DNAzymes and transfection of antisense RNA expression vectors. Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (For example, see Goldman et al., 1997).

In a still further aspect of the invention there is provided the use of an isolated DNA molecule which is the complement of a DNA molecule of the invention and which encodes an RNA molecule that hybridizes with the mRNA encoding a mutant sodium channel beta-1 subunit of the invention, in the manufacture of a medicament for the treatment of epilepsy as well as other disorders associated with sodium channel dysfunction.

In some instances, an appropriate approach for treatment may be combination therapy. This may involve the administering an antibody or complement to a mutant SCN1B subunit or sodium channel of the invention to inhibit its functional effect, combined with administration of wild-type SCN1B which may restore levels of wild-type sodium channel formation to normal levels. Wild-type SCN1B can be administered using gene therapy approaches as described above for complement administration.

There is therefore provided a method of treating epilepsy as well as other disorders associated with sodium channel dysfunction comprising administration of an antibody or complement to a mutant SCN1B subunit or sodium channel of the invention in combination with administration of wild-type SCN1B.

In still another aspect of the invention there is provided the use of an antibody or complement to a mutant SCN1B subunit or sodium channel of the invention in combination with the use of wild-type SCN1B, in the manufacture of a medicament for the treatment of epilepsy as well as other disorders associated with sodium channel dysfunction.

In a further aspect, a suitable agonist or modulator may include peptides, phosphopeptides or small organic or inorganic compounds that can restore wild-type activity of the sodium channel containing mutations in the beta-1 subunit as described above.

Peptides, phosphopeptides or small organic or inorganic compounds suitable for therapeutic applications may be identified using nucleic acids and peptides of the invention in drug screening applications as described below. Molecules identified from these screens may also be of therapeutic application in affected individuals carrying other sodium channel gene mutations, or individuals carrying mutations in genes other than sodium channels, if the molecule is able to correct the common underlying functional deficit imposed by these mutations and those of the invention.

There is therefore provided a method of treating epilepsy, as well as other disorders associated with sodium channel dysfunction, in individuals with sodium channel mutations or individuals carrying mutations in genes other than sodium channels, comprising administering a compound that is a suitable agonist or modulator of a sodium channel and that has been identified using the mutant sodium channel subunits of the invention.

In further embodiments, any of the agonists, antagonists, modulators, antibodies, complementary sequences or vectors of the invention may be administered alone or in combination with other appropriate therapeutic agents. Selection of the appropriate agents may be made by those skilled in the art, according to conventional pharmaceutical principles. The combination of therapeutic agents may act synergistically to effect the treatment or prevention of epilepsy. Using this approach, therapeutic efficacy with lower dosages of each agent may be possible, thus reducing the potential for adverse side effects.

Any of the therapeutic methods described above may be applied to any subject in need of such therapy, including, for example, mammals such as dogs, cats, cows, horses, rabbits, monkeys, and most preferably, humans.

Drug Screening

According to still another aspect of the invention, peptides of the invention, particularly purified mutant sodium channel beta-1 subunit polypeptide and cells expressing these, are useful for the screening of candidate pharmaceutical compounds in a variety of assays.

Still further, it provides the use of a polypeptide or a polypeptide complex for the screening of candidate pharmaceutical agents.

Still further, it provides the use wherein high throughput screening techniques are employed.

Compounds that can be screened in accordance with the invention include, but are not limited to peptides (such as soluble peptides), phosphopeptides and small organic or inorganic molecules (such as natural product or synthetic chemical libraries and peptidomimetics).

In one embodiment, a screening assay may include a cell-based assay utilising eukaryotic or prokaryotic host cells that are stably transformed with recombinant molecules expressing the polypeptides or fragments of the invention, in competitive binding assays. Binding assays will measure the formation of complexes between a mutated sodium channel beta-1 subunit polypeptide or fragment and the compound being tested, or will measure the degree to which a compound being tested will interfere with the formation of a complex between a mutated sodium channel beta-1 subunit polypeptide or fragment and a known ligand.

The invention is particularly useful for screening compounds by using the polypeptides of the invention in transformed cells, transfected or injected oocytes, or animal models bearing mutated sodium channel beta-1 subunits such as transgenic animals or gene targeted (knock-in) animals (see below). Drug candidates can be added to cultured cells that express a mutant SCNLB subunit (a wild-type sodium channel alpha subunit should also be expressed), can be added to oocytes transfected or injected with both a mutant SCN1B subunit and a wild-type sodium channel alpha subunit, or can be administered to an animal model containing a mutant SCN1B subunit. Determining the ability of the test compound to modulate mutant sodium channel activity can be accomplished for example by measuring the effect on the current of the channel (e.g. sodium ion flux) as compared to the current of a cell or animal containing the wild-type sodium channel. Current in cells can be measured using the patch-clamp technique (methods described in Hamill et al, 1981) or using fluorescence based assays as are known in the art (see Gonzalez et al. 1999). Drug candidates that alter the current to a more normal level are useful for treating or preventing epilepsy as well as other disorders associated with sodium channel dysfunction.

Another technique for drug screening provides high-throughput screening for compounds having suitable binding affinity to the mutant sodium channel beta-1 subunit polypeptides or sodium channels containing these (see PCT published application WO84/03564). In this stated technique, large numbers of small peptide test compounds can be synthesised on a solid substrate (such as a micotitre plate) and can be assayed for mutant SCN1B subunit polypeptide (alone or in complex with a sodium channel alpha subunit) binding. Bound mutant sodium channel or mutant SCN1B subunit polypeptide is then detected by methods well known in the art. In a variation of this technique, purified polypeptides of the invention can be coated directly onto plates to identify interacting test compounds.

The invention also contemplates the use of competition drug screening assays in which neutralizing antibodies capable of specifically binding the mutant sodium channel compete with a test compound for binding thereto. In this manner, the antibodies can be used to detect the presence of any peptide that shares one or more antigenic determinants of the mutant sodium channel.

The polypeptides of the present invention may also be used for screening compounds developed as a result of combinatorial library technology. This provides a way to test a large number of different substances for their ability to modulate activity of a polypeptide. A substance identified as a modulator of polypeptide function may be peptide or non-peptide in nature. Non-peptide “small molecules” are often preferred for many in viva pharmaceutical applications. In addition, a mimic or mimetic of the substance may be designed for pharmaceutical use. The design of mimetics based on a known pharmaceutically active compound (“lead” compound) is a common approach to the development of novel pharmaceuticals. This is often desirable where the original active compound is difficult or expensive to synthesise or where it provides an unsuitable method of administration. In the design of a mimetic, particular parts of the original active compound that are important in determining the target property are identified. These parts or residues constituting the active region of the compound are known as its pharmacophore. Once found, the pharmacophore structure is modelled according to its physical properties using data from a range of sources including x-ray diffraction data and NMR. A template molecule is then selected onto which chemical groups which mimic the pharmacophore can be added. The selection can be made such that the mimetic is easy to synthesise, is likely to be pharmacologically acceptable, does not degrade in vivo and retains the biological activity of the lead compound. Further optimisation or modification can be carried out to select one or more final mimetics useful for in vivo or clinical testing.

It is also possible to isolate a target-specific antibody and then solve its crystal structure. In principle, this approach yields a pharmacophore upon which subsequent drug design can be based as described above. It may be possible to avoid protein crystallography altogether by generating anti-idiotypic antibodies (anti-ids) to a functional, pharmacologically active antibody. As a mirror image of a mirror image, the binding site of the anti-ids would be expected to be an analogue of the original receptor. The anti-id could then be used to isolate peptides from chemically or biologically produced peptide banks.

Compounds identified through screening procedures as described above, and which are based on the use of the mutant nucleic acid and polypeptides of the invention, can also be tested for their effect on correcting the functional deficit imposed by other gene mutations in affected individuals including other sodium channel subunit mutations.

Such compounds form a part of the present invention, as do pharmaceutical compositions containing these and a pharmaceutically acceptable carrier.

Pharmaceutical Preparations

Compounds identified from screening assays and shown to restore sodium channel wild-type activity can be administered to a patient at a therapeutically effective dose to treat or ameliorate epilepsy as well as other disorders associated with sodium channel dysfunction. A therapeutically effective dose refers to that amount of the compound sufficient to result in amelioration of symptoms of epilepsy.

Toxicity and therapeutic efficacy of such compounds can be determined by standard pharmaceutical procedures in cell cultures or experimental animals. The data obtained from these studies can then be used in the formulation of a range of dosages for use in humans.

Pharmaceutical compositions for use in accordance with the present invention can be formulated in a conventional manner using one or more physiological acceptable carriers, excipients or stabilisers which are well known. Acceptable carriers, excipients or stabilizers are nontoxic at the dosages and concentrations employed, and include buffers such as phosphate, citrate, and other organic acids; antioxidants including absorbic acid; low molecular weight (less than about 10 residues) polypeptides; proteins, such as serum albumin, gelatin, or immunoglobulins; binding agents including hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitrol or sorbitol; salt-forming counterions such as sodium; and/or nonionic surfactants such as Tween, Pluronics or polyethylene glycol (PEG).

The formulation of pharmaceutical compositions for use in accordance with the present invention will be based on the proposed route of administration. Routes of administration may include, but are not limited to, inhalation, insufflation (either through the mouth or nose), oral, buccal, rectal or parental administration.

Diagnostic Applications

Polynucleotide sequences of the invention may be used for the diagnosis of epilepsy as well as other disorders associated with sodium channel dysfunction, and the use of the nucleic acid molecules of the invention in diagnosis of these disorders, is therefore contemplated.

In another embodiment of the invention, the polynucleotides that may be used for diagnostic purposes include oligonucleotide sequences, genomic DNA and complementary RNA and DNA molecules. The polynucleotides may be used to detect and quantitate gene expression in biological samples. Genomic DNA used for the diagnosis may be obtained from body cells, such as those present in the blood, tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, hybridisation using specific oligonucleotides, PCR mapping, RNAse protection, and various other methods may be employed. For instance direct nucleotide sequencing of amplification products from the sodium channel subunits of patients can be employed. Sequence of the sample amplicon is compared to that of the wild-type amplicon to determine the presence (or absence) of nucleotide differences. In addition, restriction enzyme digest and mapping can be employed for the specific C to T mutation in the SCN1B subunit described in this invention. The C to T transition at amino acid residue 85 of this subunit destroys a CfoI restriction site. The DNA from an affected individual as well as a normal control may be amplified using oligonucleotides flanking the C to T nucleotide mutation. The amplification product may then be digested by CfoI to provide a fingerprint for comparison to the DNA fingerprint of wild-type SCN1B. The DNA from an individual containing the C to T nucleotide mutation of the present invention will not be able to be digested with this enzyme however the DNA amplified from a normal individual will digest with CfoI.

According to a further aspect of the invention there is provided the use of a polypeptide, as described above, in the diagnosis of epilepsy as well as other disorders associated with sodium channel dysfunction.

When a diagnostic assay is to be based upon mutant SCN1B proteins constituting a sodium channel, a variety of approaches are possible. For example, diagnosis can be achieved by monitoring differences in the electrophoretic mobility of normal and mutant SCN1B subunit proteins that form part of the sodium channel. Such an approach will be particularly useful in identifying mutants in which charge substitutions are present, or in which insertions, deletions or substitutions have resulted in a significant change in the electrophoretic migration of the resultant protein. Alternatively, diagnosis may be based upon differences in the proteolytic cleavage patterns of normal and mutant proteins, differences in molar ratios of the various amino acid residues, or by functional assays demonstrating altered function of the gene products.

In another aspect, antibodies that specifically bind mutant sodium channels may be used for the diagnosis of a disorder, or in assays to monitor patients being treated with agonists, antagonists or modulators of the mutant sodium channel. Antibodies useful for diagnostic purposes may be prepared in the same manner as described above for therapeutics. Diagnostic assays to detect mutant sodium channels include methods that utilize the antibody and a label to detect a mutant sodium channel in human body fluids or in extracts of cells or tissues. The antibodies may be used with or without modification, and may be labelled by covalent or non-covalent attachment of a reporter molecule.

A variety of protocols for measuring the presence of mutant sodium channels, including ELISAs, RIAS, and FACS, are known in the art and provide a basis for diagnosing epilepsy, in particular generalised epilepsy with febrile seizures plus. The expression of a mutant channel is established by combining body fluids or cell extracts taken from test mammalian subjects, preferably human, with antibody to the channel under conditions suitable for complex formation. The amount of complex formation may be quantitated by various methods, preferably by photometric means. Antibodies specific for the mutant channel will only bind to individuals expressing the said mutant channel and not to individuals expressing only wild-type channels (ie normal individuals). This establishes the basis for diagnosing the disease.

Once an individual has been diagnosed with the disorder, effective treatments can be initiated. These may include administering a selective modulator of the mutant channel or an antagonist to the mutant channel such as an antibody or mutant complement as described above. Alternative treatments include the administering of a selective agonist or modulator to the mutant channel so as to restore channel function to a normal level.

Microarray

In further embodiments, complete cDNAs, oligonucleotides or longer fragments derived from any of the polynucleotide sequences described herein may be used as probes in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents. Microarrays may be prepared, used, and analyzed using methods known in the art. (For example, see Schena et al., 1996; Heller et al., 1997).

According to a further aspect of the present invention, neurological material obtained from animal models generated as a result of the identification of specific sodium channel beta-1 subunit human mutations, particularly those disclosed in the present invention, can be used in microarray experiments. These experiments can be conducted to identify the level of expression of specific sodium channel beta-1 subunits, or any cDNA clones from whole-brain libraries, in epileptic brain tissue as opposed to normal control brain tissue. Variations in the expression level of genes, including sodium channel beta-1 subunits, between the two tissues indicates their involvement in the epileptic process either as a cause or consequence of the original sodium channel mutation present in the animal model. Microarrays may be prepared, as described above.

Transformed Hosts

The present invention also provides for the production of genetically modified (knock-out, knock-in and transgenic), non-human animal models transformed with the nucleic acid molecules of the invention. These animals are useful for the study of the function of a sodium channel, to study the mechanisms of disease as related to a sodium channel, for the screening of candidate pharmaceutical compounds, for the creation of explanted mammalian cell cultures which express a mutant sodium channel and for the evaluation of potential therapeutic interventions.

Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, genetically modified mice and rats are highly desirable due to the relative ease of generation of knock-in, knock-out and transgenic animals and their ease of maintenance and shorter life spans. For certain studies, transgenic yeast or invertebrates may be suitable and preferred because they allow for rapid screening and provide for much easier handling. For longer term studies, non-human primates may be desired due to their similarity with humans.

To create an animal model for a mutated sodium channel of the invention several methods can be employed. These include but are not limited to generation of a specific mutation in a homologous animal gene, insertion of a wild type human gene and/or a humanized animal gene by homologous recombination, insertion of a mutant (single or multiple) human gene as genomic or minigene cDNA constructs using wild type or mutant or artificial promoter elements or insertion of artificially modified fragments of the endogenous gene by homologous recombination. The modifications include insertion of mutant stop codons, the deletion of DNA sequences, or the inclusion of recombination elements (1ox p sites) recognized by enzymes such as Cre recombinase.

To create transgenic or gene targeted (knock-in) mice, which are preferred, a mutant version of a sodium channel beta-1 subunit can be inserted into a mouse germ line using standard techniques of oocyte microinjection. Alternatively, if it is desired to inactivate or replace an endogenous sodium channel beta-1 subunit gene, homologous recombination using embryonic stem cells may be applied.

For oocyte injection, one or more copies of the mutant sodium channel beta-1 subunit gene can be inserted into the pronucleus of a just-fertilized mouse oocyte. This oocyte is then reimplanted into a pseudo-pregnant foster mother. The liveborn mice can then be screened for integrants using analysis of tail DNA or DNA from other tissues for the presence of the particular human subunit gene sequence. The transgene can be either a complete genomic sequence injected as a YAC, BAC, PAC or other chromosome DNA fragment, a complete cDNA with either the natural promoter or a heterologous promoter, or a minigene containing all of the coding region and other elements found to be necessary for optimum expression.

According to still another aspect of the invention there is provided the use of genetically modified nonWO human animals as described above for the screening of candidate pharmaceutical compounds (see drug screening above). These animals are also useful for the evaluation (eg therapeutic efficacy, toxicity, metabolism) of candidate pharmaceutical compounds, including those identified from the invention as described above, for the treatment of epilepsy and/or other disorders associated with sodium channel dysfunction.

It will be clearly understood that, although a number of prior art publications are referred to herein, this reference does not constitute an admission that any of these documents forms part of the common general knowledge in the art, in Australia or in any other country.

Throughout this specification and the claims, the words “comprise”, “comprises” and “comprising” are used in a non-exclusive sense, except where the context requires otherwise.

### BRIEF DESCRIPTION OF THE DRAWINGS

FIG. 1 shows the GEFS+pedigree for the family containing the R85C mutation. Individuals containing the mutation are indicated. The original proband is marked as P.

FIG. 2 shows a sequencing trace of the SCN1B mutation identified in this study. The sequencing trace for the affected individual is represented by the top panel indicating the C→T nucleotide change, while the bottom panel shows the sequencing trace of a normal individual.

FIG. 3 shows sodium channel amino acid alignments surrounding the SCN1B mutation. The SCN1B highly conserved arginine amino acid at position 85 is boxed. This amino acid is also highly conserved in members of the immunoglobulin gene superfamily but is not conserved in other SCNB subunits.

### MODES FOR PERFORMING THE INVENTION

### EXAMPLE 1

### Clinical Diagnosis of Affected Family Members

A group of 4 family members spanning 3 generations was studied because of the occurrence of generalized epilepsy with febrile seizures plus (GEFS+) phenotypes (see FIG. 1 for pedigree).

The proband (P) was ascertained through routine clinical practice, and has an affected first degree relative and multiple affected second degree relatives. In the family members so far studied, all affected individuals have had the phenotype of FS+, with infrequent (range 5-10) febrile and afebrile tonic-clonic seizures commencing between the ages of 15 months and 2 years, and in the 2 older affected cases, ceasing by mid-childhood and the early teenage years respectively. All are intellectually normal and otherwise healthy. The extended family is known to have a large number of other individuals (approximately 13) whose phenotypes are consistent with GEFS+. There are no known consanguineous relationships in the family.

### EXAMPLE 2

### Mutation Analysis of SCN1B

Single stranded conformation polymorphism (SSCP) analysis and sequencing were performed on GEFS+affected individuals to identify disease causing mutations.

Primers used for SSCP were labelled at their 5′ end with HEX. The primers were designed within flanking SCN1B introns to enable amplification of each exon of SCN1B (Table 1 and SEQ ID Numbers:3-12). Typical PCR reactions were performed in a total volume of 10 μl using 30 ng of patient DNA. PCR reactions were performed in 96 well plates or 0.5 ml tubes depending on batch size, and contained 67 mM Tris-HCl (pH 8.8); 16.5 mM (NH₄)₂SO₄; 6.5 μM EDTA; 1.5 mM MgCl₂; 200 μM each DNTP; 10% DMSO; 0.17 mg/ml BSA; 10 mM β-mercaptoethanol; 15 μg/ml each primer and 100 U/ml Taq DNA polymerase. For exons 1, 2, 4 and 5, PCR reactions were performed using 10 cycles of 94° C. for 30 seconds, 60° C. for 30 seconds, and 72° C. for 30 seconds followed by 25 cycles of 94° C. for 30 seconds, 55° C. for 30 seconds, and 72° C. for 30 seconds. A final extension reaction for 10 minutes at 72° C. followed. For exon 3, PCR reactions were performed using 35 cycles of 94° C. for 30 seconds, 62° C. for 30 seconds, and 72° C. for 30 seconds with a final extension reaction for 10 minutes at 72° C. Twenty μl of loading dye comprising 50% (v/v) formamide, 12.5 mM EDTA and 0.02% (w/v) bromophenol blue were added to completed reactions which were subsequently run on non-denaturing 4% polyacrylamide gels with a cross-linking ratio of 35:1 (acrylamide:bis-acrylamide) and containing 2% glycerol. Gel thickness was 100 μm, width 168 mm and length 160 mm. Gels were run at 1200 volts and approximately 20 mA, at ambient temperature using the GelScan 2000 system (Corbettt Research, Australia) according to manufacturers specifications. Results were subsequently analysed using the ONE-Dscan gel analysis software package (Scanalytics Inc.).

PCR products showing a conformational change were subsequently sequenced. This first involved reamplification of the relevant amplicon using primers without the 5′ HEX addition followed by purification of the PCR amplified templates for sequencing using QiaQuick PCR preps (Qiagen) based on manufacturers procedures. The primers used to sequence the purified SCN1E amplicons were identical to those used for the initial amplification step. For each sequencing reaction, 25 ng of primer and 100 ng of purified PCR template were used. The BigDye sequencing kit (ABI) was used for all sequencing reactions according to the manufacturers specifications. The products were run on an ABI 377 Sequencer and analysed using the EditView program.

A total of 53 unrelated GEFS+patients were screened by fluorescent-SSCP analysis and sequencing. No mutations were found in 17 sporadic cases of GEFS+tested. Of the 36 families tested, 3 were found to have point mutations in SCN1A (Wallace et al. 2001), two were found to contain the C121W mutation in SCN1B (Wallace et al. 1998) and a single family (FIG. 1) identified a novel SCN1B mutation in exon 3 due to a C to T nucleotide mutation at position 253 as represented by SEQ ID NO:1. The nucleotide change results in the replacement of an arginine amino acid residue for a cysteine residue at position 85 of the encoded protein, as represented by SEQ ID NO:2. Each of these changes were not present in the control population.

TABLE 1

Primers Used for SSCP Analysis of SCN1B

| | | | Size |
|---|---|---|---|
| Exon | Forward | Reverse | (bp) |

| | | | |
|---|---|---|---|
| 1 | CGCCTCTCGCCCCGCTATTA | CTCCCGCCGCCCCGCCGTG | 169 |
| 2 | CCTGACCTGAGCCTGCTGTC | TGCCCTCCCATGCCGTTAA | 228 |
| 3 | CCTTCCCCTCCCTGGCTAC | GGCAGGCAGCACCCGACTCA | 285 |
| 4 | CAGCCTGGGCTACCCCCTTA | CCCTGGGTGCCCTCCCACCT | 220 |
| 5 | CGGTCTGATGATGGGGTCAC | TTACGGCTGGCTCTTCCTTG | 243 |

### EXAMPLE 3

### Characteristics of The Novel SCN1B Mutation

The C to T nucleotide change of the present invention results in the destruction of the CfoI restriction enzyme site. This provides a means for diagnosing epilepsy in individuals containing this mutation. For example DNA obtained from the individual to be tested can be amplified with primers as used in the SSCP analysis of exon 3 (SEQ ID Numbers: 7 and 8). The resultant amplified DNA can subsequently be purified and digested with the restriction enzyme CfoI. Amplimers that contain the C to T nucleotide change will not be digested by this enzyme whereas the amplimer generated from wild-type individuals will be reduced in size compared to that of an affected individual due to digestion of the amplimer.

In both the C121W and R85C SCN1B mutations, the amino acid substitution occurs in the extracellular amino-terminal domain of the protein. Voltage-gated sodium channel beta subunits (beta-1, beta-2) are integral membrane proteins having a single transmembrane region and a prominent extracellular amino-terminal domain. Recombinant sodium channel beta-1 subunits have been demonstrated to exert significant modulatory effects on the gating behaviour and expression levels of various alpha subunits, including brain isoforms (Isom et al. 1992; Makita et al. 1994). The functional effects of the beta-1 subunit on gating modulation are dependant on structures located primarily in the extracellular domain (McCormick et al. 1998; Makita et al. 1996). The extracellular domain contains a single immuno-globulin like fold motif bearing a high degree of amino-acid similarity to corresponding regions of a neural cell adhesion molecule (contactin) and other members of the immunoglobulin gene superfamily (Isom et al. 1995; McCormick et al. 1998). This motif is structurally maintained by a single putative disulfide bridge between two highly conserved cysteine residues, including Cysl21 in SCN1B. Therefore the C121W mutation is likely to disrupt the disulfide bridge which may alter the secondary structure of the extracellular domain. Interestingly, the novel SCN1B mutation which constitutes a preferred embodiment of the present invention (R85C) replaces an arginine residue with a cysteine residue. The presence of this cysteine residue may affect the functioning of the ion-channel through interference with normal disulfide bridge formation or other, as yet undetermined, mechanisms.

The genetic heterogeneity of GEFS+is now well established. Screening of SCN1B and SCN1A in a panel of 53 patients indicates that no mutations were found in the 17 isolated cases of GEFS+. However, the frequency of GEFS+causing mutations in each of these genes in the remaining 36 familial cases was −17% (6/36). The low proportion of GEFS+cases caused by these two sodium channel genes indicates that other genes are involved. Obvious candidates include other neuronal sodium channels and proteins that interact with sodium channels. The confirmation that SCN1B is one of the many genes likely to be responsible for febrile seizures raises the possibility of greater diagnostic accuracy, more precise genetic counselling and a more rational basis for treatment for febrile seizures and generalised epilepsy.

### EXAMPLE 4

### Analysis of Mutant SCN1B Subunits and Sodium Channels Incorporating These

The following methods are used to determine the structure and function of mutant SCN1B subunits and sodium channels incorporating these.

Molecular Biological Studies

The ability of a mutant sodium channel of the invention as a whole or through individual mutant beta-1 subunits to bind known and unknown proteins can be examined. Procedures such as the yeast two-hybrid system are used to discover and identify any functional partners. The principle behind the yeast two-hybrid procedure is that many eukaryotic transcriptional activators, including those in yeast, consist of two discrete modular domains. The first is a DNA-binding domain that binds to a specific promoter sequence and the second is an activation domain that directs the RNA polymerase II complex to transcribe the gene downstream of the DNA binding site. Both domains are required for transcriptional activation as neither domain can activate transcription on its own. In the yeast two-hybrid procedure, the gene of interest or parts thereof (BAIT), is cloned in such a way that it is expressed as a fusion to a peptide that has a DNA binding domain. A second gene, or number of genes, such as those from a cDNA library (TARGET), is cloned so that it is expressed as a fusion to an activation domain. Interaction of the protein of interest with its binding partner brings the DNA-binding peptide together with the activation domain and initiates transcription of the reporter genes. The first reporter gene will select for yeast cells that contain interacting proteins (this reporter is usually a nutritional gene required for growth on selective media). The second reporter is used for confirmation and while being expressed in response to interacting proteins it is usually not required for growth.

The nature of the sodium channel interacting genes and proteins can also be studied such that these partners can also be targets for drug discovery.

Structural Studies

SCN1B recombinant proteins of the invention can be produced in bacterial, yeast, insect and/or mammalian cells and used in crystallographical and NMR studies. Together with molecular modeling of the proteins as well as modeling of sodium channels incorporating these, structure-driven drug design can be facilitated.

### EXAMPLE 5

### Generation of Polyclonal Antibodies

Antibodies can be made to selectively bind and distinguish mutant SCN1B protein from wild-type protein. Antibodies specific for mutagenised epitopes are especially useful in cell culture assays to screen for cells which have been treated with pharmaceutical agents to evaluate the therapeutic potential of the agent.

To prepare polyclonal antibodies, short peptides can be designed homologous to the SCN1B amino acid sequence. Such peptides are typically 10 to 15 amino acids in length. These peptides should be designed in regions of least homology to other receptor subunits and should also have poor homology to the mouse orthologue to avoid cross species interactions in further down-stream experiments such as monoclonal antibody production. Synthetic peptides can then be conjugated to biotin (Sulfo-NHS-LC Biotin) using standard protocols supplied with commercially available kits such as the PIERCE™ kit (PIERCE). Biotinylated peptides are subsequently complexed with avidin in solution and for each peptide complex, 2 rabbits are immunized with 4 doses of antigen (200 μg per dose) in intervals of three weeks between doses. The initial dose is mixed with Freund's Complete adjuvant while subsequent doses are combined with Freund's Immuno-adjuvant. After completion of the immunization, rabbits are test bled and reactivity of sera is assayed by dot blot with serial dilutions of the original peptides. If rabbits show significant reactivity compared with pre-immune sera, they are then sacrificed and the blood collected such that immune sera can be separated for further experiments.

Antibodies to mutant beta-1 subunits can be used to detect the presence and the relative level of the mutant forms in various tissues.

### EXAMPLE 6

### Generation of Monoclonal Antibodies

Monoclonal antibodies can be prepared in the following manner. Immunogen comprising an intact mutant SCN1B subunit protein or mutant SCN1B subunit peptide is injected in Freund's adjuvant into mice with each mouse receiving four injections of 10 to 100 μg of immunogen. After the fourth injection blood samples taken from the mice are examined for the presence of antibody to the immunogen. Immune mice are sacrificed, their spleens removed and single cell suspensions are prepared (Harlow and Lane, 1988). The spleen cells serve as a source of lymphocytes, which are then fused with a permanently growing myeloma partner cell (Kohler and Milstein, 1975). Cells are plated at a density of 2×10⁵ cells/well in 96 well plates and individual wells are examined for growth. These wells are then tested for the presence of GABA receptor subunit specific antibodies by ELISA or RIA using wild type or mutant subunit target protein. Cells in positive wells are expanded and subcloned to establish and confirm monoclonality. Clones with the desired specificity are expanded and grown as ascites in mice followed by purification using affinity chromatography using Protein A Sepharose, ion-exchange chromatography or variations and combinations of these techniques.

References:

References cited herein are listed on the following pages, and are incorporated herein by this reference.

Alekov, A K. et al. (2000). J. Physiol. 529: 533-539.

Annegers, J F. (1996). The Treatment of epilepsy: Principles and practice. Second Edition. (Wyllie E (Ed) Williams and Wilkins).

Baulac, S. et al. (1999). Am. J. Hum. Genet. 65: 1078-1085.

Berkovic, S F. et al. (1987). Neurology 37: 993-1000.

Berkovic, S F. Et al. (1994). The epilepsies: specific syndromes or a neurobiological continuum? In: Epileptic seizures and syndromes (Wolf (Ed) London: John Liddey). 25-37.

Commission on Classification and Terminology of the international League Against Epilepsy. (1989). Epilepsia. 30: 389-399.

Escayg, A. et al. (2000). Mature Genet. 24: 343-345.

Gardiner, M. (2000). J. Neurol. 247: 327-334.

Gonzalez, J E. et al. (1999). Drug Discov. Today 4: 431-439.

Hamill, O P. et al. (1981). Pflugers Arch. 391: 85-100.

Harlow, E. and Lane, D. (1988). Antibodies: A Laboratory Manual (Cold Spring Harbor Laboratory, Cold Spring Harbor, N.Y.).

Huse, W D. et al. (1989). Science 246: 1275-1281.

Isom, LL. et al. (1992). Science 256: 839-842.

Isom, L L. et al. (1995). Cell 83: 433-442.

Kohler, G. and Milstein, C. (1975). Nature 256: 495-497.

Lopes-Cendes, I. et al. (2000). Am. J. Hum. Genet. 66: 698-701.

Makita, N. et al. (1994). J. Biol. Chem. 269: 7571-7578.

Makita N. et al. (1996). J. Neurosci. 16: 7117-7127.

McCormick, K A. et al. (1998). J. Biol. Chem. 273: 3954-3962.

Moulard, B. et; al. (1999). Am. J. Hum. Genet. 65: 1396-1400.

Peiffer, A. et al. (1999). Ann. Neurol. 46: 671-678.

Reutens, D C. and Berkovic, S F. (1995). Neurology 45: 1469-1476.

Roger, J. et al. (1992). Epileptic syndromes in infancy, childhood and adolescence. Second Edition. (John Libbey, London).

Scheffer, I E. et al. (2000). Ann. Neurol. 47: 840-841.

Scheffer, I E. and Berkovic, SF. (1997). Brain 120: 479-490.

Singh, R. et al. (1999). Ann Neurol. 45: 75-81.

Sutton, GC. (1990). The principles and practice of medical genetics. Second Edition. (Churchill Livingstone, N.Y.).

Wallace, R H. et al. (2001). Am. J. Hum. Genet. 68: 859865.

Wallace, R H. et al. (1998). Nature Genet. 19: 366-370.

1. An isolated nucleic acid molecule encoding a mutant mammalian beta-1 subunit of a voltage-gated sodium channel wherein a mutation event has occurred and said mutation event disrupts the functioning of an assembled sodium channel so as to produce an epilepsy phenotype, with the proviso that said mutation event is not one which results in a C121W substitution in the encoded polypeptide.

2. An isolated nucleic acid molecule as claimed in claim 1 wherein said mutation event results in the introduction of a cysteine residue in the extracellular loop of the amino terminal domain of the encoded polypeptide.

3. An isolated nucleic acid molecule as claimed in claim 2 wherein said mutation event takes place in exon 3.

4. An isolated nucleic acid molecule as claimed in claim 3 wherein said mutation event occurs at position 253 of the coding sequence.

5. An isolated nucleic acid molecule as claimed in claim 4 wherein said mutation event is a C to T nucleotide substitution.

6. An isolated nucleic acid molecule comprising the nucleotide sequence set forth in SEQ ID NO:1.

7. An isolated nucleic acid molecule consisting of the nucleotide sequence set forth in SEQ ID NO:1.

8. An isolated mammalian polypeptide, said polypeptide being a mutant mammalian beta-1 subunit of a voltage-gated sodium channel wherein a mutation event has occurred and said mutation event disrupts the functioning of an assembled sodium channel so as to produce an epilepsy phenotype, with the proviso that said mutation event is not a C121W substitution.

9. An isolated polypeptide as claimed in claim 8 wherein said mutation event introduces a cysteine residue to the extracellular loop of the amino terminal domain of the polypeptide.

10. An isolated polypeptide as claimed in claim 8 wherein said mutation event occurs at position 85.

11. An isolated polypeptide as' claimed in claim 10 wherein said mutation event is an R85C substitution.

12. An isolated polypeptide comprising the amino acid sequence set forth in SEQ ID NO:2.

13. An isolated polypeptide consisting of the amino acid sequence set forth in SEQ ID NO:2.

14. An isolated polypeptide complex, said polypeptide complex being an assembled mammalian voltage-gated sodium channel in which a mutation event has occurred in the beta-1 subunit and said mutation event disrupts the functioning of the assembled sodium channel so as to produce an epilepsy phenotype, with the proviso that said mutation event is not a C121W substitution.

15. An isolated polypeptide complex, as claimed in claim 14 wherein said mutation event introduces a cysteine residue to the extracellular loop of the amino terminal domain of the beta-1 subunit.

16. An isolated polypeptide complex as claimed in claim 15 wherein said mutation event occurs at position 85 in the beta-1 subunit.

17. An isolated polypeptide complex as claimed in claim 16 wherein said mutation event is an R85C substitution.

18. A cell transformed with an isolated nucleic acid molecule as claimed in any one of claims 1 to 7.

19. A cell comprising mammalian sodium channels incorporating a mutant beta-1 subunit as defined in any one of claims 8 to 11.

20. A method of preparing a polypeptide, comprising the steps of:
(1) culturing cells as claimed in claim 18 or 19 under conditions effective for polypeptide production; and
(2) harvesting the polypeptide.

21. A polypeptide prepared by the method of claim 20.

22. An antibody which is immunologically reactive with an isolated polypeptide as claimed in any one of claims 8 to 13, a polypeptide as claimed in claim 21, or an isolated polypeptide complex as claimed in any one of claims 14-17.

23. An antibody as claimed in claim 22 which is selected from the group consisting of a monoclonal antibody, a humanised antibody, a chimaeric antibody or an antibody fragment including a Fab fragment, (Fab′)₂ fragment, Fv fragment, single chain antibodies and single domain antibodies.

24. A method of treating epilepsy as well as other disorders associated with sodium channel dysfunction, comprising administering a selective agonist, antagonist or modulator of the sodium channel when it has undergone a mutation event as defined in any one of claims 8 to 11 or 14 to 17 to a subject in need of such treatment.

25. The use of a selective agonist, antagonist or modulator of the sodium channel when it has undergone a mutation event as defined in any one of claims 8 to 11 or 14 to 17 in the manufacture of a medicament for the treatment of epilepsy as well as other disorders associated with sodium channel dysfunction.

26. A method of treating epilepsy as well as other disorders associated with sodium channel dysfunction comprising administering an antibody as claimed in either one of claims 22 or 23 to a subject in need of such treatment.

27. A method of treating epilepsy as well as other disorders associated with sodium channel dysfunction, comprising administering an isolated DNA molecule which is the complement (antisense) of a nucleic acid molecule as defined in any one of claims 1 to 7 and which encodes an RNA molecule that hybridizes with the mRNA encoding a mutant sodium channel beta-1 subunit, to a subject in need of such treatment.

28. The use of a DNA molecule which is the complement of a nucleic acid molecule as defined in any one of claims 1 to 7 and which encodes an RNA molecule that hybridizes with the mRNA encoding a mutant sodium channel beta-1 subunit, in the manufacture of a medicament for the treatment of epilepsy as well as other disorders associated with sodium channel dysfunction.

29. A method of treating epilepsy as well as other disorders associated with sodium channel dysfunction, comprising administering an antibody, as claimed in either one of claims 22 or 23 or a DNA molecule which is the complement of a nucleic acid molecule as defined in any one of claims 1 to 7 and which encodes an RNA molecule that hybridizes with the mRNA encoding a mutant sodium channel beta-1 subunit, in combination with administration of wild-type SCN1B, to a subject in need of such treatment.

30. The use of an antibody, as claimed in claims 22 or 23 or a DNA molecule which is the complement of a nucleic acid molecule as defined in any one of claims 1 to 7 and which encodes an RNA molecule that hybridizes with the mRNA encoding a mutant sodium channel beta-1 subunit, in combination with the use of wild-type SCN1B, in the manufacture of a medicament for the treatment of epilepsy as well as other disorders associated with sodium channel dysfunction.

31. Use of a polypeptide as claimed in any one of claims 8 to 13 or a polypeptide complex as claimed in any one of claims 14 to 17 for the screening of candidate pharmaceutical agents.

32. Use as claimed in claim 31 wherein high throughput screening techniques are employed.

33. A method of screening for a modulator of sodium channel activity useful in the treatment of epilepsy as well as other disorders associated with sodium channel dysfunction comprising the steps of:
a) providing a cell as claimed in claim 18 or 19;
b) contacting said cell with a test compound; and
c) detecting if said test compound binds the encoded protein or modulates the biological activity of a sodium channel incorporating the encoded protein;
wherein a test compound that binds the protein or modulates biological activity of a sodium channel incorporating the encoded protein is a compound useful for the treatment of the disorder.

34. A method of screening for a modulator of sodium channel activity useful in the treatment of epilepsy as well as other disorders associated with sodium channel dysfunction comprising the steps of:
a) providing a polypeptide as claimed in any one of claims 8 to 13 or a polypeptide complex as claimed in any one of claims 14-17;
b) contacting said polypeptide or polypeptide complex with a test compound; and
c) detecting if said test compound binds the polypeptide or polypeptide complex;
wherein a test compound that binds the polypeptide or polypeptide complex is a compound useful for the treatment of the disorder.

35. A compound when identified by a method as claimed in either one of claims 33 or 34.

36. A pharmaceutical composition comprising a compound as claimed in claim 35 and a pharmaceutically acceptable carrier.

37. A genetically modified non-human animal transformed with an isolated nucleic acid molecule as defined in any one of claims 1 to 7.

38. A genetically modified non-human animal as claimed in claim 37 in which the animal is selected from the group consisting of rats, mice, hamsters, guinea pigs, rabbits, dogs, cats, goats, sheep, pigs and non-human primates such as monkeys and chimpanzees.

39. The use of a genetically modified non-human animal as claimed in claim 37 or 38 in the screening of candidate pharmaceutical compounds.

40. An expression vector comprising a DNA molecule as claimed in any one of claims 1 to 7.

41. The use of a DNA molecule as claimed in any one of claims 1 to 7 in the diagnosis of epilepsy, in particular generalised epilepsy with febrile seizures plus, and other disorders associated with sodium channel dysfunction.

42. The use of a polypeptide as defined in any one of claims 8 to 17 in the diagnosis of epilepsy, in particular generalised epilepsy with febrile seizures plus, as well as other disorders associated with sodium channel dysfunction.

43. The use of an antibody as defined in claim 22 or 23 in the diagnosis of epilepsy, in particular generalised epilepsy with febrile seizures plus, as well as other disorders associated with sodium channel dysfunction.
